(19) United States
(12) Patent Application Publication
(10) Pub. No.: US 2021/0207131 A1
(43) Pub. Date: Jul. 8, 2021

(54) Multiplex Alteration of Cells Using a Pooled Nucleic Acid Library and Analysis Thereof

(71) Applicant: President and Fellows of Harvard College, Cambridge, MA (US)

(72) Inventors: George M. Church, Brookline, MA (US); Eswar Prasad Ramachandran Iyer, Somerville, MA (US); Conor Camplisson, San Marcos, CA (US)

(21) Appl. No.: 15/999,615

(22) PCT Filed: Feb. 17, 2017
(86) PCT No.: PCT/US17/18322

§ 371 Date: Aug. 20, 2018

Related U.S. Application Data
(60) Provisional application No. 62/296,746, filed on Feb. 18, 2016.

(51) Int. Cl. C12N 15/10

(52) U.S. Cl. CPC C12N 15/1079; C12N 15/1065

(57) ABSTRACT

A method of altering cells with a pooled library of bar-coded nucleic acids is provided including combining a group of cells with the pooled library of bar coded nucleic acids under conditions which promote uptake of one or more barcoded nucleic acids from the pooled library into the cells, analyzing an individual cell for phenotype, sequencing the individual cell to identify the one or more barcoded nucleic acids from the pooled library, and correlating the phenotype for the individual cell with the one or more barcoded nucleic acids from the pooled library.

### STATEMENT OF GOVERNMENT INTERESTS

This invention was made with government support under 5P50HG005550-05 awarded by NIH/NHRGRI. The government has certain rights in the invention.

### FIELD

The present invention relates in general to methods of altering cells using a pooled library of nucleic acids, determining phenotype of the altered cells and sequencing of the altered cells to determine genotype.

### BACKGROUND

Methods of altering cells and measuring biological activity are known.

### SUMMARY

The disclosure provides a method of altering cells with a pooled library of bar coded nucleic acids including combining a group of cells with the pooled library of bar coded nucleic acids under conditions which promote uptake of one or more barcoded nucleic acids from the pooled library into the cells, wherein the barcodes are functional or nonfunctional, analyzing an individual cell for phenotype, sequencing the individual cell to identify the one or more barcoded nucleic acids from the pooled library, and correlating the phenotype for the individual cell with the one or more barcoded nucleic acids from the pooled library. The disclosure provides that the steps of analyzing, sequencing and correlating are conducted for multiple cells within the group of cells. The disclosure provides that one or more barcoded nucleic acids from the pooled library are delivered to the cells using viral or non-viral methods. The disclosure provides that the individual cell is analyzed for phenotype using automated imaging and one or more barcoded nucleic acids within the individual cell is sequenced using FISSEQ. The disclosure provides that the cells are analyzed for phenotype using high-content imaged based analysis and one or more barcoded nucleic acids within the individual cell is sequenced using FISSEQ. The disclosure provides that the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype. The disclosure provides that the one or more barcoded nucleic acids are amplified in situ before using FISSEQ. The disclosure provides that the one or more barcoded nucleic acids are amplified in situ using isothermal or non-isothermal amplification before using FISSEQ. The disclosure provides that the nucleic acid from the library element is delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using FISSEQ.

The disclosure provides a method of altering cells with a pooled library of drugs including combining a group of cells with the pooled library of drugs under conditions which promote uptake of one or more drugs from the pooled library into the cells, analyzing an individual cell for phenotype, analyzing the individual cell to identify the one or more drugs from the pooled library, and correlating the phenotype for the individual cell with the one or more drugs from the pooled library.

The disclosure provides a method of altering cells with a pooled library of nucleic acids having fluorescent barcodes including combining a group of cells with the pooled library of nucleic acids having fluorescent barcodes under conditions which promote uptake of one or more nucleic acids having fluorescent barcodes from the pooled library into the cells, analyzing an individual cell for phenotype, analyzing the individual cell to identify the fluorescent barcode, and correlating the phenotype for the individual cell with the fluorescent barcode.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts in schematic a flow chart describing the pooled library image screen methods.

FIG. 2A and FIG. 2B depict in schematic contructs used in the pooled library image screen methods.

FIG. 3 depicts images of results of sgRNA targeted reverse transcription.

### DETAILED DESCRIPTION

The present disclosure provides methods of altering a plurality of cells with a pooled library of nucleic acids. One or more nucleic acids of the pooled library are introduced into the cells. The cells are analyzed for phenotype. Nucleic acids within the cells are sequenced. The cell phenotype is correlated with the one or more nucleic acids from the pooled library. The one or more nucleic acids identified from sequencing results in the observed phenotype.

The disclosure provides a library of nucleic acids. Such a library of nucleic acids may be generated by in silico pooled oligonucleotide synthesis technologies to generate large genomic libraries, such as for screens such as for pooled selection screens. See Wang, T., Wei, J. J., Sabatini, D. M. & Lander, E. S. Genetic screens in human cells using the CRISPR-Cas9 system. Science 343, 80-84 (2014); Gilbert, L. A. et al. Genome-Scale CRISPR-Mediated Control of Gene Repression and Activation. Cell 159, 647-661 (2014); Shalem, O., Sanjana, N. E. & Zhang, F. High-throughput functional genomics using CRISPR-Cas9. Nat Rev Genet 16, 299-311 (2015); Shalem, O. et al. Genome-scale CRISPR-Cas9 knockout screening in human cells. Science 343, 84-87 (2014) each of which is hereby incorporated by reference in its entirety.

The disclosure provides synthesis of the library of nucleic acids using methods known to those of skill in the art. The library or subcomponents thereof, is delivered to the cells, such as by methods known to those in the art such as viral and non-viral methods. Such methods include lentiviral transduction. The disclosure provides that the cells may be cultured, such as under selective pressure and lysed and sequencing, such as deep-sequencing, may be performed to identify one or more nucleic acids from the library that are introduced into the cells using methods known to those of skill in the art. See Shalem, O., Sanjana, N. E. & Zhang, F. High-throughput functional genomics using CRISPR-Cas9. Nat Rev Genet 16, 299-311 (2015); Shalem, O. et al. Genome-scale CRISPR-Cas9 knockout screening in human cells. Science 343, 84-87 (2014); Agrotis, A. & Ketteler, R. A new age in functional genomics using CRISPR/Cas9 in arrayed library screening. Front Genet 6, 300 (2015) each of which is hereby incorporated by reference in its entirety. Statistically-overrepresented nucleic acids may be determined using methods associated with pooled and arrayed screens. See Agrotis, A. & Ketteler, R. A new age in functional genomics using CRISPR/Cas9 in arrayed library screening. Front Genet 6, 300 (2015) hereby incorporated by reference in its entirety.

The disclosure provides analysis of the cells to determine phenotype using methods known to those of skill in the art. Automated microscopy based High Content Screening (HCS) measures biological activity in single cells or whole organisms that have been subjected to libraries of compound or nucleic acids, such as a chemical compound library, an siRNA library, a guide RNA library, as ORFeome library and the like. See Buchser, W. et al. Assay Guidance Manual. (2004); Bray, M. A. & Carpenter, A. Assay Guidance Manual. (2004) each of which is hereby incorporated by reference in its entirety.

The disclosure provides a method of pooled library image screening including screening cells with a pooled library of compounds, such as nucleic acids. The cells are analyzed for phenotype. The cells are analyzed to determine presence on or within the cells of one or more members of the library of compounds. The phenotype of a cell is correlated with the identification of the one or more members of the library of compounds on or within a cell. The disclosure provides a method of a pooled screen, cytological profiling and in situ sequencing. See Lee, J. H. et al. Fluorescent in situ sequencing (FISSEQ) of RNA for gene expression profiling in intact cells and tissues. Nat Protoc 10, 442-458 (2015); Lee, J. H. et al. Highly multiplexed subcellular RNA sequencing in situ. Science 343, 1360-1363 (2014); Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015); Ke, R. et al. In situ sequencing for RNA analysis in preserved tissue and cells. Nat Methods 10, 857-860 (2013) each of which are hereby incorporated by reference in its entirety.

The disclosure provides that a pooled library of nucleic acids is delivered to cells using physical, chemical, viral or non-viral methods using methods known to those of skill in the art. See Nayerossadat, N., Maedeh, T. & Ali, P. A. Viral and nonviral delivery systems for gene delivery. Adv Biomed Res 1, 27 (2012) hereby incorporated by reference in its entirety. One or more of the cells, such as each cell, randomly uptakes single or multiple library elements, i.e. one or more of the members of the library. The biological activity, i.e. phenotype, resulting from the introduction of the one or more of the members of the library on or into the cell is measured, such as with high precision using automated imaging and high-content image-based analysis. See Carpenter, A. E. et al. CellProfiler: image analysis software for identifying and quantifying cell phenotypes. Genome Biol 7, R100 (2006) hereby incorporated by reference in its entirety. The cells are then subjected to analysis of the presence on or within the cell of one or more library elements using methods known to those of skill in the art. An exemplary method is sequencing of nucleic acids within the cell, such as spatially-resolved in-situ sequencing which is used to identify unique library elements uptaken by individual cells. See Crosetto, N., Bienko, M. & van Oudenaarden, A. Spatially resolved transcriptomics and beyond. Nat Rev Genet 16, 57-66 (2015) hereby incorporated by reference in its entirety. Other methods such as spatially resolved sequencing methods may be used. See Lee, J. H. et al. Fluorescent in situ sequencing (FISSEQ) of RNA for gene expression profiling in intact cells and tissues. Nat Protoc 10, 442-458 (2015); Lee, J. H. et al. Highly multiplexed subcellular RNA sequencing in situ. Science 343, 1360-1363 (2014); Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015); Ke, R. et al. In situ sequencing for RNA analysis in preserved tissue and cells. Nat Methods 10, 857-860 (2013); Larsson, C., Grundberg, I., Söderberg, O. & Nilsson, M. In situ detection and genotyping of individual mRNA molecules. Nat Methods 7, 395-397 (2010); Weibrecht, I. et al. In situ detection of individual mRNA molecules and protein complexes or post-translational modifications using padlock probes combined with the in situ proximity ligation assay. Nat Protoc 8, 355-372 (2013); Chen, R. et al. A Barcoding Strategy Enabling Higher-Throughput Library Screening by Microscopy. ACS Synth Biol 4, 1205-1216 (2015) each of which are hereby incorporated by reference in its entirety. Useful aspects of identification methods include high sensitivity of detection of nucleic acid barcodes, high signal-to-noise to allow rapid imaging at lower magnification objective to allow fast data collection, scalable aspects and cost-effectiveness. Exemplary methods include spatially-resolved in-situ sequencing methods using padlock probes (see Ke, R. et al. In situ sequencing for RNA analysis in preserved tissue and cells. Nat Methods 10, 857-860 (2013) hereby incorporated by reference in its entirety); multiplexed error-robust fluorescence in situ hybridization (MERFISH; see Chen et al., Science; 2015; 348 (6233) aaa6090, doi: 10.1126/science.aaa6090 hereby incorporated by reference in its entirety); and fluorescence in-situ sequencing (FISSEQ; Lee, J. H. et al. Fluorescent in situ sequencing (FISSEQ) of RNA for gene expression profiling in intact cells and tissues. Nat Protoc 10, 442-458 (2015); Lee, J. H. et al. Highly multiplexed subcellular RNA sequencing in situ. Science 343, 1360-1363 (2014) each of which is hereby incorporated by reference in its entirety.)

Data or information related to biological activity is correlated with data or information regarding presence on or within the cell of one or more library elements using methods known to those of skill in the art. The disclosure provides bioinformatic methods that are used to merge cytometric data with sequence information at the single cell level. The disclosure provides statistical deconvolution of the effect of each library element or pertubant with resulting phenotype. The disclosure provides methods with the sensitivity of arrayed formats (where each perturbing agent is physically or fluidically separated) and with the mutiplexability and cost-reduction of pooled screens.

The disclosure provides a method of pooled library imaging screening using a single vessel including the library of components (which may include on the order of from 1000 to 1,000,000 library components) and the cells (which may include 1000 cells to 10,000,000 cells).

The disclosure provides amplification or pre-amplification methods that may be used with sequencing methods described herein. The disclosure provides PCR-based amplification or pre-amplification of target nucleic acid sequences. Such amplification or pre-amplification may be isothermal or non-isothermal. In-situ PCR (see Bagasra, O. Protocols for the in situ PCR-amplification and detection of mRNA and DNA sequences. Nat Protoc 2, 2782-2795 (2007); Mitra, R. D. & Church, G. M. In situ localized amplification and contact replication of many individual DNA molecules. Nucleic Acids Res 27, e34 (1999) each of which is hereby incorporated by reference in its entirety) or similar methods may be used to amplify a barcoded region of a nucleic acid of the library several orders of magnitude, such that the sequences become available for more ready detection.

The disclosure provides biological amplification and targeted reverse transcription for use with in situ sequencing. Genomic libraries are introduced into cells in an expression vector where a high number of RNA copies are produced within each cell, such as by using a promoter, such as U6, to create a large number of RNA copies within each cell. These RNA molecules, which themselves may serve a biological function (CRISPR, ORF etc.) also serve as barcodes, i.e. “expressed barcodes” which may be targeted for reverse transcription using a common set of primers that bind to a sequence found commonly in all library elements.

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, bacteria cells, archael cells, eubacterial cells and the like. Cells include eukaryotic cells such as yeast cells, plant cells, and animal cells. Particular cells include mammalian cells and human cells. Particular cells include stem cells, such as pluripotent stem cells, such as human induced pluripotent stem cells.

Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety.

Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

In certain aspects of the invention, vectors and plasmids useful for transformation of a variety of host cells are provided. Vectors and plasmids are common and commercially available from companies such as Invitrogen Corp. (Carlsbad, Calif.), Stratagene (La Jolla, Calif.), New England Biolabs, Inc. (Beverly, Mass.) and Addgene (Cambridge, Mass.).

Certain aspects of the invention pertain to vectors, such as, for example, expression vectors. As used herein, the term “vector” refers to a nucleic acid sequence capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. By way of example, but not of limitation, a vector of the invention can be a single-copy or multi-copy vector, including, but not limited to, a BAC (bacterial artificial chromosome), a fosmid, a cosmid, a plasmid, a suicide plasmid, a shuttle vector, a P vector, an episome, YAC (yeast artificial chromosome), a bacteriophage or viral genome, or any other suitable vector. The host cells can be any cells, including prokaryotic or eukaryotic cells, in which the vector is able to replicate.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IPL, IPR, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

In certain exemplary embodiments, the recombinant expression vectors comprise a nucleic acid sequence in a form suitable for expression of the nucleic acid sequence in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the foreign nucleic acid sequence encoding a plurality of ribonucleic acid sequences described herein is linked to the regulatory sequence(s) in a manner which allows for expression of the nucleic acid sequence. The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). It will be appreciated by those skilled in the art that the design of the expression vector can depend on such factors as the choice of the host cell to be transformed, the level of expression of protein desired, and the like.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, insect cells, fungal cells, archaeal cells, eubacterial cells, a virion, a virosome, a virus-like particle, a parasitic microbe, an infectious protein and the like. Cells include eukaryotic cells such as yeast cells, plant cells, and animal cells. Particular cells include bacterial cells. Other suitable cells are known to those skilled in the art.

Foreign nucleic acids (i.e., those which are not part of a cell's natural nucleic acid composition) may be introduced into a cell using any method known to those skilled in the art for such introduction. Such methods include transfection, transduction, infection (e.g., viral transduction), injection, microinjection, gene gun, nucleofection, nanoparticle bombardment, transformation, conjugation, by application of the nucleic acid in a gel, oil, or cream, by electroporation, using lipid-based transfection reagents, or by any other suitable transfection method. One of skill in the art will readily understand and adapt such methods using readily identifiable literature sources.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IP_{L}, IP_{R}, T7, tac, and trc (useful for expression in Escherichia coli and Pseudomonas); the amy, apr, npr promoters and various phage promoters useful for expression in Bacillus subtilis, and Bacillus lichenformis; nisA (useful for expression in gram positive bacteria, Eichenbaum et al. Appl. Environ. Microbiol. 64(8):2763-2769 (1998)); and the synthetic P1I promoter (useful for expression in Lactobacillus plantarum, Rud et al., Microbiology 152:1011-1019 (2006)). Termination control regions may also be derived from various genes native to the preferred hosts.

According to certain aspect of the invention, phages and their genetic material are provided. As used herein, the terms “phage” and “bacteriophage” are used interchangeably. Phage can be distinguished from each another based on their genetic composition and/or their virion morphology. Some phage have double stranded DNA genomes, including phage of the corticoviridae, lipothrixviridae, plasmaviridae, myrovridae, siphoviridae, sulfolobus shibate, podoviridae, tectiviridae and fuselloviridae families. Other phage have single stranded DNA genomes, including phage of the microviridae and inoviridae families. Other phage have RNA genomes, including phage of the leviviridae and cystoviridae families. Exemplary bacteriophage include, but are not limited to, Wphi, Mu, T1, T2, T3, T4, T5, T6, T7, P1, P2, P4, P22, fd, phi6, phi29, phiC31, phi80, phiX174, SP01, M13, MS2, PM2, SSV-1, L5, PRD1, Qbeta, lambda, UC-1, HK97, HK022 and the like.

Exemplary libraries useful in the disclosed methods include CRISPR libraries such as those at world wide website addgene.org/crispr/libraies; human ORFeome such as those at world wide website dharmacon.gelifesciences.com/gene-expression-cdnas-orfs/mammalian-orfs/horfeome-v81-library; siRNA libraries, microRNA libraries, phagemid antibody libraries, ribosome display libraries and the like. Other libraries useful in the methods described herein will become apparent to those of skill in the art based on the present disclosure.

Guide RNA as described herein refer to the guide RNA component of a CRISPR system known to those of skill in the art. The guide RNA includes a spacer sequence of about 20 nucleotides which is complementary to a target nucleic acid sequence, referred to as a protospacer.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Pooled Library Image Screen (PLIS)

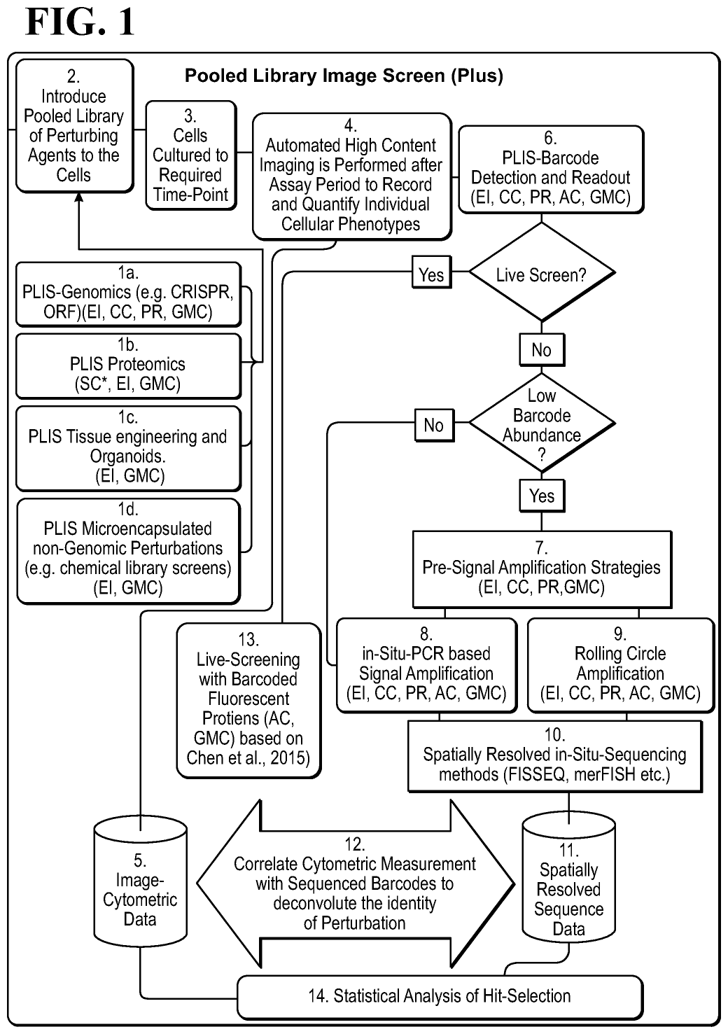

The disclosure provides a method of pooled library image screening as generally depicted in FIG. 1. Pooled and barcoded libraries can be introduced inside biological systems (e.g. cells, iPSC derived human organoids or whole organism) using a suitable physical, chemical or biological method such that each cell discretely and probabilistically uptakes one to several library elements based upon the delivery conditions (See FIG. 1, steps 1a-1c and step 2.). Cells are cultured until the required time-point (See FIG. 1, step 3). The resulting phenotypic consequence of the introduced library element is assayed via high-content microscopic analysis (See FIG. 1, step 4 where automated high content imaging is performed after the assay period to record and quantify individual cellular phenotype). Images are segmented and analyzed to identify and measure cytometric changes in each cell to obtain cytometric data (See FIG. 1, step 5). In order to identify the library element introduced into each cell, targeted and/or un-targeted in-situ-sequencing is used to identify barcodes and in addition can be used to simultaneously measure transcriptomic changes (See FIG. 1, step 6). In cases that require pre-amplification of a target nucleic acid sequence such as when a barcode is in low abundance, either in-situ PCR based signal amplification or rolling circle amplification or other amplification methods known to those of skill in the art can be used to amplify the target nucleic acid sequence (See FIG. 1, steps 7-9). Spatially resolved In-situ sequencing methods known to those of skill in the art (e.g. FISSEQ, MERFISH) is performed on the same cells to read the introduced barcodes associated with each perturbation event (See FIG. 1, step 10) and spatially resolved sequence data is collected (See FIG. 1, step 11). Sequenced reads are computationally mapped to cellular and sub-cellular compartments identified from step 5. Cytometric measurements or data from step 5 are correlated with sequence barcodes from step 11 to deconvolute the identity of perturbation, i.e. the phenotype is matched with the library element on or within the cell. (See FIG. 1, step 12.) Bioinformatic methods or pipelines are used to perform statistical analyses and hit-selection (See FIG. 1, step 14).

### Example II

### Common Sequences in Expressed Libraries are Used in Targeted In Situ Sequencing

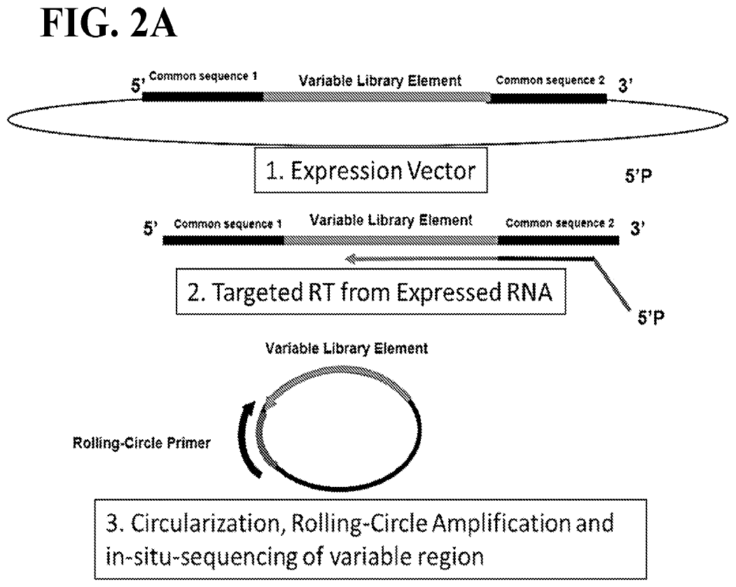

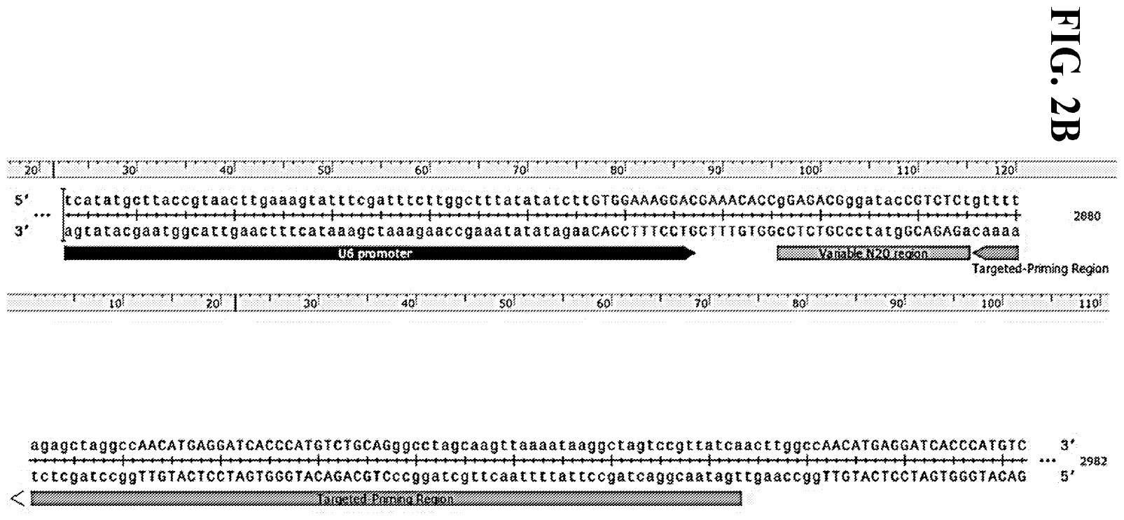

Library elements can be introduced into the cells as expression plasmids which contain a high-expressivity promoter and express several RNA copies of a variable library region, such as a spacer sequence for guide RNA (CRISPR N20) or an ORFeome. As depicted in FIG. 2A, the disclosure provides an expression vector (step 1) which includes a variable library element having a 5′ first common sequence (common sequence 1) and a 3′ second common sequence (common sequence 2). The first common sequence and the second common sequence flank the variable library element. As depicted in FIG. 2A, the common sequences in the expressed RNA can be used to perform targeted-cDNA conversion (step 2) using universal RT Primers containing FISSEQ adapters (red) to generate linear cDNA molecules containing both the variable region and FISSEQ adapter. In FIG. 2A, step 3, the cDNA molecules are enzymatically circularized using CircLigase and amplified using rolling circle amplification to generate hundreds of tandem copies of library sequence, which can then be sequenced with FISSEQ.

The disclosure provides an example of a vector using a 20 nucleotide spacer sequence of a guide RNA as a variable region. As depicted in FIG. 2B, sgRNA has 20-nucleotide variable region (Orange) at the 5′ end that determines the DNA target site, and also contains a common backbone region (green). Targeted-RT is performed using universal primers targeting the common-region (green), and cDNA molecules containing the variable N20 region are circularized and sequenced using FISSEQ. The variable also serves as an expressed barcode and is used to map the identity of perturbation within a given cell.

### Example III

### Transfection of a Pooled Library of CRISPR sgRNA in Human PGP1 Fibroblasts

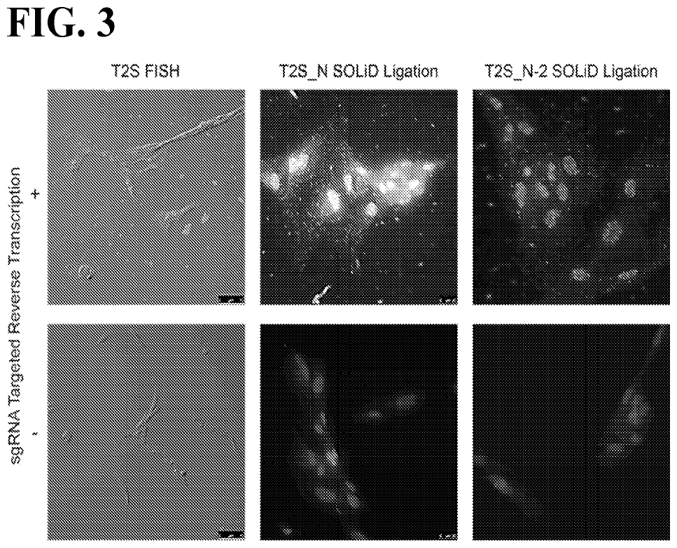

A pooled-library of CRISPR sgRNA was transfected in human PGP1 fibroblasts and a high-density of targeted rolling circle amplicons were generated (See FIG. 3), compatible with FISSEQ for sequencing and detection. The same approach was used to detect the ORFeome library. One advantage of this approach is that it does not require additional modifications to the plasmid or vectors, and is compatible with existing libraries, as long as there is a common sequence to perform targeted-RT. Targeted primers were designed for the experiments using methods known to those of skill in the art.

The disclosure provides a Pooled Library Image Screen using targeted amplification and sequencing of a Synergistic Activation Mediator (SAM) library sgRNA in primary human fibroblasts from the Personal Genome Project (PGP1F). Cells were transiently transfected using a lentivirus with the pooled SAM sgRNA library including over 70,290 guide spacer sequences targeting all human RefSeq coding isoforms. See Konermann, S. et al. Genome-scale transcriptional activation by an engineered CRISPR-Cas9 complex. Nature 517, 583-588 (2015) hereby incorporated by reference in its entirety. The reverse transcription primer targets expressed sgRNAs and adds an adapter sequence (T2S) which in turn serves as a common priming site for amplification, fluorescent in situ hybridization (FISH), and sequencing using FISSEQ. FISH was used to verify successful amplification and to quantify amplicon density. Using the T2S_N sequencing primer, the first two bases of the targeted sequence are interrogated. Amplicons appear in all four channels due to the diversity of the SAM sgRNA library. Using the T2S_N-2 sequencing primer, the last two bases of the T2S adapter sequence are interrogated, which are the same for every amplicon, which serves as a sequencing control.

The disclosure provides that a pooled library of sgRNA or ORFeome can be lentivirally delivered to cultured cells. Using targeted spatially-resolved sequencing (as described above), the identity of each library element can be deconvoluted (as shown in FIG. 2). The disclosure provides targeted generation of rolling-circle products from PGP1 fibroblast cells transiently transfected with Synergistic activation mediator (SAM) sgRNA library consisting of over 70,000 unique guide RNA sequences.

### Example IV

### Micropatterned Cells

The disclosure provides methods of cell micropattering in combination with High Content Screening. See Harkness, T. et al. High-Content Imaging with Micropatterned Multiwell Plates Reveals Influence of Cell Geometry and Cytoskeleton on Chromatin Dynamics. Biotechnol J (2015). doi:10.1002/biot.201400756 hereby incorporated by reference in its entirety. Pooled library image screening is combined with cell-micropatterning to perform highly-sensitive pooled library image screens.

The disclosure provides a method to perform pooled library imaging screening on engineered human organs. An EB-Grid is combined with pooled library imaging screening to create gene deletions (or expression controls) of key regulatory genes which result in the development of fully vascularized organoids or systems of organs, without forming a human embryo. The gridded organ-systems are used for Protein, RNA or drug-library screens with high-content imaging and/or FISSEQ. The specified layout of the organs in a 3D pattern enable reproducibility, imaging and easy data interpretation.

### Example V

### Pooled Image Screens on Live Cells

The disclosure provides methods of performing pooled library image screening using fluorescent proteins for barcoding in live cells. See FIG. 1, step 13 and Chen, R. et al. A Barcoding Strategy Enabling Higher-Throughput Library Screening by Microscopy. ACS Synth Biol 4, 1205-1216 (2015) hereby incorporated by reference in its entirety. The use of fluorescent reports allows pooled library image screening to be performed on live cells, without in-situ sequencing.

### Example VI

### Pooled Library Image Drug Screening

The disclosure provides methods of pooled library image screening using drugs. Chemical compounds are encapsulated or coated on micro or nanoparticles (see Bao, G., Mitragotri, S. & Tong, S. Multifunctional nanoparticles for drug delivery and molecular imaging. Annu Rev Biomed Eng 15, 253-282 (2013) hereby incorporated by reference in its entirety) along with a nucleic acid barcode such as a DNA barcode, and delivered to cells or tissues, in culture or whole organisms. Pooled library image screening is used to deconvolute the identity of compounds uptaken by cells, and the resulting phenotype.

### Example VII

### High-Throughput Cell and Tissue Engineering

The disclosure provides methods of pooled library image screening to create in parallel large number of genomic variants in single cells or engineered organoids, which can be used to perform pooled forward/reverse genetic screens. The disclosure provides that mutant human iPSCs can be parallel generated with PLIS, to screen for factors that allow growth of 3-D vascularized human organoids. For example, human iPSC cells exposed to genomic library are dissociated and cultured in matrigel in low density to permit them to grow into 3-dimentional embryioid bodies and organoids. See Meinhardt, A. et al. 3D reconstitution of the patterned neural tube from embryonic stem cells. Stem Cell Reports 3, 987-999 (2014). 3-D phenotypes are identified via screening and the identity of the pertubing library element is deconvoluted with in-situ sequencing. This process is iterated to identify factor promoting growth of desired vascularized tissue type. This process screens for factors that can enable the growth of vascularized organoids.

### Example VII

### Materials and Methods

Cell Culture and Lentiviral Transduction: Cells are cultured under standard culture conditions. Brifefly, PGP1 Fibroblasts, or HeLa-Cas9 cells, are cultured in DMEM with 4.5 g/L D-glucose, GlutaMAX™ Supplement, and 110 mg/L sodium pyruvate, supplemented with 10% FBS (Gibco) and 1% penicillin-streptomycin. Cells are seeded on glass-bottom dishes (MatTek) and subsequently transduced with a lentiviral library containing genetic perturbations (sgRNAs, TFs, etc.). In order to perform statistically analysis, the cell seeding and viral titer delivery should be optimized to yield at least 10-100× coverage (each library element is represented in at least 10-100 cells). Optionally, drug selection can be performed to eliminate cells that were not transduced.

in situ Sequencing Library Construction: Once lentiviral transduction and selection are complete. cDNA libraries are constructed for in situ sequencing. Cells are fixed with 4% paraformaldehyde for 15 minutes, permeabilized with 70% ethanol for 5 minutes, and treated with 0 IN HCl for 1 minute A targeted reverse transcription primer with common adapter sequence is then used to capture expressed library elements (e.g. sgRNA molecules). Reverse transcription (RT), circularization, and rolling circle amplification (RCA) are performed as previously described in Lee J H, Daugharthy E R, Scheiman J, Kalhor R. Yang J L, Ferrante T C, et al. Highly multiplexed subcellular RNA sequencing in situ. Science. 2014 Mar. 21,343(6177):1360-3, and Lee J H Daugharthy E R. Scheiman J, Kalhor R. Ferrante T C. Terry R, et al. Fluorescent in situ sequencing (FISSEQ) of RNA for gene expression profiling in intact cells and tissues Nat Protoc 2015 March:10(3)-442-58 each of which is hereby incorporated by reference in its entirety (See Appendix 1: FISSEQ Library Construction Protocol). Alternatively, in situ amplification and sequencing can be accomplished using Padlock Probes (see Weibrecht 1, Lundin E. Kiflemariam S, Mignardi M, Grundberg I, Larsson C, et al. In situ detection of individual mRNA molecules and protein complexes or post-translational modifications using padlock probes combined with the in situ proximity ligation assay. Nat Protoc. 2013 February;8(2):355-72; Larsson C, Grundberg I, Soderberg O, Nilsson M In situ detection and genotyping of individual mRNA molecules. Nat Methods 2010 May; 7(5):395-7 each of which ar hereby incorporated by reference in its entirety (see Appendix 2: Padlock Probe Amplification Protocol) or Polony in sinu PCR which combines Polony PCR (see Mitra R D. Church G M In situ localized amplification and contact replication of many individual DNA molecules. Nucleic Acids Res. 1999 Dec. 15:27(24).e34 hereby incorporated by reference in its entirety with in-situ PCR (see Bagasra O. Protocols for the in situ PCR-amplification and detection of mRNA and DNA sequences. Nat Protoc 2007:2(11):2782-95 hereby incorporated by reference in its entirety. In brief, cells are fixed, permeabilized and embedded in a layer of gel (for example Acrylamide). The primers are optionally cross-linked to the gel and in-situ PCR amplification if performed (see Appendix 3: Polony Amplification Protocol).

Primers used:

| | |
|---|---|
| T2S-IDT- | /5Phos/ACTTCAGCTGCCCCGGGTGAAGATGATA |
| gRNA- | ACGGACTAGC [HPLC][order 250 |
| RT- | nmole] |
| Primer-1 | |
| T2S-WT- | /5Phos/ACTTCAGCTGCCCCGGGTGAAGACTAGC |
| gRNA- | CTTATTTTAA [HPLC][order 250 |
| RT- | nmole] |
| Primer-2 | |
| gRNA_Cy3_Probe | /5Cy3/AACTTTGCTATTTCTAGCTCTAAAAC |
| (AKA gRNA- | [HPLC][order 250 nmole] |
| FISH-probe) | |
| T2S_IDT_gRNA_ | GTTTTAGAGCTAGAAATAGCA*A*G |
| RCA_P1 | |
| gRNA- | /5Phos/AACTTGCTATTTCTAGCTCTAAAAC |
| Sequencing | |
| Anchor | |

Fluorescent in situ Sequencing: Once the library preparation is complete, the quality of sequencing libraries is verified by fluorescence in situ hybridization (FISH). For example, a fluorescently labeled oligo-probe complementary to an adapter sequence added during reverse transcription. This provides the density and subcellular localization of amplicons. The probe is then stripped and replaced with a sequencing primer. After successful library construction, either SOLiD (see Appendix 4: SOLiD Sequencing Protocol) or fluorescent 9-mer sequencing by ligation (SBL) chemistries can be used to determine identity of the sgRNA present in each cell (see 7. Ke R, Mignardi M, Pacureanu A. Svedlund J, Botling J, Wählby C, et al. In situ sequencing for RNA analysis in preserved tissue and cells. Nat Methods. 2013 September; 10(9):857-60 hereby incorporated by reference in its entirety (see Appendix 5: Sequencing by ligation using 9-mer probes). Sequencing can be performed with either manual or automated fluidic exchanges.

Cytological Profiling. The goal of this step is to identify the corresponding phenotypic changes associated with modification by a library element. Once the library construction is successfully completed, the sample is mounted on a microscope with appropriate settings. In order to measure the phenotypic state of each cell, the cells are subjected to phenotypic screening. This can be performed prior to sequencing, or immediately post sequencing. However it is exemplary that the different image sets are perfectly aligned within the X-Y-Z coordinates to enable cell data merging. In order to achieve this, the samples cannot be moved once they are mounted on the microscopes until high-content screening and sequencing both have taken place. The cells are subjected to standard high-content image based screening assays based on the biological problem of interest. For example, tagged proteins, small molecules and antibodies can be used to label and image the cellular features of interest (see Buchser W, Collins M Garyantes T, Guha R, Haney S. Lemmon V. et al. Assay Development Guidelines for Image-Based High Content Screening. High Content Analysis and High Content Imaging. In: Sittampalam G S, Coussens N P, Nelson H. Arkin M. Auld D. Austin C, et al., editors. Assay Guidance Manual [Internet] Bethesda (Md.): Eli Lilly & Company and the National Center for Advancing Translational Sciences; 2004: Gustafsdottir S M. Ljosa V, Sokohicki K L, Anthony Wilson J. Walpita D Kemp M M. et al. Multiplex cytological profiling assay to measure diverse cellular states. PLoS ONE. 2013 Dec. 2; 8(12):e80999 each of which is hereby incorporated by reference in its entirety (see Appendix 6. Sample protocol for cell staining).

Image Processing and Bioinformatics Pipeline: Images are passed through a custom bioinformatics pipeline which aligns images, performs base calling. For example the images can be analyzed using CellProfiler software (see Bray M-A, Vokes Miss., Carpenter A E. Using CellProfiler for Automatic Identification and Measurement of Biological Objects in Images Curr Protoc Mol Biol. 2015 Jan. 5.109:14.17.1-14.17.13. Carpenter A E, Jones T R, Lamprecht M R, Clarke C, Kang I H, Friman O, et al. CellProfiler: image analysis software for identifying and quantifying cell phenotypes Genome Biol. 2006 Oct. 31; 7(10):R100 each of which are hereby incorporated by reference in its entirety. Briefly illumination correction, background subtraction and feature segmentation is performed. The nuclei, cytoplasm and other stained features are identified. The image analysis pipeline can then measure various parameters like cell size, shape, texture, intensity, local density of object of interest within certain cellular compartments (eg. nucleus vs. cytoplasm). Images are also processed to identify and read the barcoded sequences using another pipeline (an example pipeline is described in Lee J H, Daugharthy E R, Scheiman J, Kalhor R, Ferrante T C, Terry R, et al. Fluorescent in situ sequencing (FISSEQ) of RNA for gene expression profiling in intact cells and tissues. Nat Protoc. 2015 March; 10(3):442-58 hereby incorporated by reference in its entirety. The identified barcodes are eventually mapped at single cell level to individual cellular and subcellular compartments identified from the first image-processing pipeline, since both datasets are aligned in common X-Y-Z coordinates.

### APPENDIX 1: FISSEQ LIBRARY CONSTRUCTION PROTOCOL

Protocols from Lee et al. 2014 may be used for the remaining steps of the library construction protocol after reverse transcription. After this protocol, the sample is ready for sequencing. For Example, Lee et al 2014 provides that the sample is washed using 1×PBS and cross-linked using BS(PEG)9 (Thermo Scientific), diluted to 50 mM in PBS, for 1 hour at 25° C. 1 M Tris (G Biosciences) is added to quench the reaction for 30 minutes at 25° C. A mixture of DNase-free RNases (Roche Diagnostics) and RNase H (Enzymatics) is added to degrade residual RNA for 1 hour at 37° C. A 100 uL circularization reaction mixture (Ix reaction buffer, 2.5 mM MnCl2, 1 M Betaine and 5 uL CircLigase II from Illumina/Epicentre) is then added to the sample well and incubated at 60° C. for 2 hours. After circularization, the sample is washed using H20 and incubated with a 200 uL mixture containing 0.1 uM RCA primer (TCTTCAGCGTTCCCGA*G*A from IDT) in 2×SSC and 30% formamide for 15 minutes at 60° C. The sample is washed using 2×SSC, and a 200 uL amplification mixture containing 500 U 3 Phi29 DNA polymerase (Enzymatics), 250 uM dNTP, 40 uM, and aminoallyl dUTP is added. The sample is incubated in a dry 30° C. chamber overnight and cross-linked using BS(PEG)9 diluted to 50 mM in PBS for 1 hour at 25° C. After a rinse with PBS, 1 M Tris is added to quench the reaction for 30 minutes.

### APPENDIX 2: PADLOCK PROBE AMPLIFICATION PROTOCOL

Protocols from Larsson et al. 2010 provide an alternative strategy for generating RCA amplicons, via the hybridization, ligation, and amplification of padlock probes. After this protocol, the sample is ready for sequencing. For example, Larsson et al. provides that to make the target cDNA strands available for padlock probe hybridization, the RNA portion of the created RNA-DNA hybrids was degraded with ribonuclease H. This was performed in the same step as the padlock probe hybndization and ligation. For most reactions, Ampligase (Epicentre) was used for ligation. Samples were first preincubated in Ampligase buffer (20 mM Tris-HCl, pH 8 3, 25 mM KCl, 10 mM MgCl₂, 0.5 mM NAD and 0.01% Triton X-100). Ligation was then carried out with 100 nM of each padlock probe in a mix of 0.5 U μl⁻¹ Ampligase, 0.4 U μl⁻¹ RNase H (Fermentas), 1 U μl⁻¹ RiboLock RNase Inhibitor. Ampligase buffer, 50 mM KCl and 20% formamide. Incubation was performed first at 37° C. for 30 min, followed by 45 min at 45° C. For detection of actin transcript isoforms in mouse embryonic tissue sections, ligation was instead carried out using T4 DNA ligase (Fennentas). Samples were then first preincubated in T4 DNA ligase buffer (Fermentas). Then 100 nM of each padlock probe was added with 0.1 U μl⁻¹ T4 DNA ligase, 0.4 U μl⁻¹ RNase H, 1 U μl⁻¹ Ribolock RNase Inhibitor and 0.2 μg μl⁻¹ BSA in T4 DNA ligase buffer supplemented with 0.5 mM ATP and 250 mM NaCl. Slides were then incubated at 37° C. for 30 mm. After ligation with Ampligase or T4 DNA ligase, slides were washed in DEPC-treated 2×SSC with 0.05% Tween-20 at 37° C. for 5 min and rinsed in PBS-T Slides were preincubated briefly in Φ29 DNA polymerase buffer (Fermentas). RCA was then performed with 1 U μl⁻¹ Φ029 DNA polymerase (Fermentas) in the supplied reaction buffer, 1 U μl⁻¹ RiboLock RNase Inhibitor, 250 μM dNTPs, 0.2 μg μl⁻¹BSA and 5% glycerol. Incubation was carried out for 60 min at 37° C. The incubation was followed by a wash in PBS-T.”

### APPENDIX 3: POLONY AMPLIFICATION PROTOCOL

An alternate exemplary protocol for generating amplicons wherein modified PCR primers are covalently crosslinked to a polyacrylamide gel matrix, fixing them in space is provided below. See also see (Mitra and Church, 1999). After this protocol, the sample is ready for sequencing.

Cast Acrylamide Gels
1. Remove 8 Bind-Silane treated slides from storage box in dessicator and place face up in AirClean hood.
2. Turn on UV lamp in AirClean hood for 15 minutes.
3. Label slides with numbers and date using a SHARPIE pen (other inks will wash off in hexane).
4. Almost completely cover slides with coverslips (Fisherbrand, 18 mm×30 mm, #1, untreated). Keep a small area of oval exposed for subsequent gel loading.
5. Prepare fresh “ABD mix” in a 15 ml polystyrene conical:
   9 mL IEF 40% Acrylamide
   1 mL Acrylamide/Bis (19:1; 38%:2%)
   200 mg DATD
6. Using a 3-cc syringe and a 0.22 micron filter, filter ˜1 mL of the ABD mix into a 1.5 mL microcentrifuge tube.
7. Remove Acrydite-Modified Amplification Primer from pre-PCR area of −20° C. freezer to thaw.
8. Prepare fresh 5% APS. Notably, the APS bottle should be stored a room-temperature dessicator such as a large plastic screw-top container.
   0.5 g APS→10 mL dH20
9. Prepare fresh 5% TEMED
   2 uL TEMED→38 uL dH20.
10. Put a few drops of 30% BSA into a 1.5 mL tube (BSA should be stored at 4′C)
11. Prepare the gel-casting mix (200 uL total volume). Do not add APS until immediately prior to casting the gels. This recipe is for a 10% gel with sufficient mix for at least 8 (identical) slides.

| | | |
|---|---|---|
| 50 | uL | A-B-D mix (FILTERED) |
| 1.33 | uL | 30% BSA |
| 2 | uL | JCF-AC (100 uM) |
| 136.66 | uL | dH20 |
| 4 | uL | 5% TEMED |
| 4 | uL | 5% APS |
| 0.5 | uL | Template (at appropriate concentration) |

12. Gel Loading:
Suck up 18 microliters of the mix and pipet it into the small exposed area of the oval, such that surface tension pulls the liquid into the space between the coverslip and glass to cover the full surface area of the oval. The liquid should distribute under the coverslip such that only a small amount (1 to 2 uL) cannot fit. Slide the coverslip over such that the oval is completely covered.
As many as 8 gels from a single mix are loaded (after adding APS). For more slides, the master mix may be split prior to adding APS (so that polymerization doesn't proceed too far in the tube before all of the gels are cast). The master-mix can also be split prior to adding APS if needed to have a different template on each slide, for instance.
13. Place the slides on a flat tray and load the tray into the argon chamber (and fill with argon). Allow gels to polymerize for ˜30 minutes.
14. Remove slides from argon chamber. Use a razor-blade to cleanly remove cover-slips from polymerized gels by sticking the edge of the blade under the coverslip and gently “popping” it up.
15. To wash off the excess acrylamide monomer, place slides in a dH20 filled plastic Coplin jar and incubate for 30 minutes at RT with slow shaking.
16. Remove slides from Coplin jar and place face up in PCR hood.

Diffuse in PCR Reagents
17. Prepare diffuse-in mix while allowing slides to dry, while not allowing the slides to over-dry. A thin, shrinking film of liquid on the surface of each gel may be observed. Generally, the diffuse-in mix is added within 5 minutes or so of this shrinking film's disappearance. This generally means about 30 minutes of drying.
18. Prepare diffuse-in mix (200 uL total volume):

| | | |
|---|---|---|
| 152.33 | uL | dH20 |
| 10 | uL | 5 mM unlabeled dNTP mix |
| 20 | uL | 10x Taq Buffer (with MgC12) |
| 1.33 | uL | 30% BSA |
| 2 | uL | 10% Tween-20 |
| 1 | uL | unmodified amplification primer (100 uM) |
| 13.33 | uL | Jumpstart TAQ (2.5 units/uL) |

19. Pipet 25 uL of diffuse-in mix to the center of each gel. Assuming the gel is newly dried, the liquid will ball up rather than spreading over the surface.
20. Apply 18×30 mm cover-slip by resting one edge of the coverslip on the Teflon coat and allowing the coverslip to “fall” onto the gel. The liquid should spread to evenly cover the surface of the gel.
21. Apply an orange SecureSeal chamber. Seal down the edges with the blunt end of a set of tweezers.
22. Fill chamber with mineral oil. Use a P1000 pipetman filled with ˜550 uL of mineral oil. Use a Chemwipe to dry off the tip of the pipet tip.
23. Wipe off any excess mineral oil from the surface of the SecureSeal, if necessary. Seal holes.

PCR Amplification
24. Place slides in thermocycler (with labels facing out).
25. To PCR, cycle slides as follows:
   1. 94 C×3 minutes
   2. 94 C×45 seconds
   3. 58 C×30 seconds
   4. 72 C×3 minutes
   5. Goto Step 2, 43 more times
   6. 72 C×6 minutes
   7. 4 C forever
Both the annealing temperature and the extension time can be adjusted to optimize for the set of amplification primers being used and the length of the PCR products being formed, respectively. The above protocol may be used for an 800 bp template.

Slide Clean Up
26. Fill a GLASS Coplin jar with Hexane
27. Fill a PLASTIC Coplin jar with Wash 1E
28. Without removing cover-slips, place the slides in the hexane-filled glass Coplin jar and leave for 5 minutes.
29. One-by-one, remove the slides and cleanly pop off coverslips with a razor-blade as above. Quick wave in the air is sufficient to evaporate off residual hexane. Remove all residual adhesive from the cover-chamber using a razor-blade, and place the slide in the plastic Coplin containing Wash 1E.
30. Wash 2×4′ in Wash 1E (meaning, 4 minutes shaking in Wash 1E, then replace the solution in the same Coplin jar with fresh Wash 1E and allow it to go another 4 minutes on the shaker).
31. Gels can remain indefinitely (at least a week or two) before proceeding.

### APPENDIX 4: SOLID SEQUENCING PROTOCOL

Protocols from Lee et al. 2014 may be used to perform SOLiD sequencing. For example, five sequencing primers are designed specific to a universal adaptor (N, N-1, N-2, N-3, N-4, where N-x is recessed at the 5′ end by x-nt). These sequencing primers are annealed to the sample sequentially, and k ligation reactions are performed for each primer (k+1 ligation reactions for primers N-2, N-3, and N-4). Each sequencing primer is annealed to the sample at 2.5 uM in 200 uL 80° C. 5×SASC (0.75 M sodium acetate and 75 mM tri-sodium citrate, pH 7.5), incubating for 10 minutes at 25° C. The sample is washed twice for one minute each with 1 mL 1× Instrument Buffer (SOLiD Instrument Buffer Kit, Applied Biosystems Cat #4389784). 200 uL sequencing mix is freshly prepared on ice using 165 uL nuclease-free H20, 20 uL T4 DNA ligase buffer, 10 uL T4 DNA Ligase (Enzymatics), and 5 uL SOLiD sequencing oligos (the dark purple tubes from the SOLiD ToP Sequencing Kit Fragment Library F3 Tag MM50 Cat #4449388). After aspirating the Instrument Buffer, the sequencing mix is added to the sample and incubated at 25° C. for 45 minutes. The sequencing mix is aspirated, and the sample is washed with 1× Instrument Buffer (four 1 mL washes for 5 minutes each). Imaging is done in 1 mL 1× Instrument Buffer. After aspirating the Instrument Buffer, the fluorophore is cleaved to allow for subsequent ligation. The sample is incubated twice for 5 minutes each in 200 uL 1× Cleave Solution 1 (SOLiD ToP Instrument Buffer Kit Component 4406489), followed by two incubations for five minutes each in 200 uL 1× Cleave Mix 2.1 (SOLiD ToP Instrument Buffer Kit Component 4445677, prepared fresh with 106.7 uL Cleave 4 Solution 2.1 Part 1 and 293.3 uL Cleave Solution Part 2). After the second incubation with Cleave Mix 2.1, the sample is washed three times for 5 minutes each with 1× Instrument Buffer. After repeating the cyclic ligation process k (or k+1) times, the ligated strands are stripped by four 5 minute washes in 80° C. strip buffer (80% formamide, 0.01% Triton-X100). Another sequencing primer is annealed, and the cyclic ligation process is repeated.”

### APPENDIX 5: SEQUENCING BY LIGATION USING 9-MER PROBES

Protocols from Ke R. et al., 2013 may be used for sequencing by ligation. For example, before the sequencing is performed, the detection probes are stripped off. The slides are first incubated through an ethanol series to remove the mounting medium and dried at room temperature. For detection probes without uracils, the samples ar first washed with DEPC-PBS-T and then incubated three times with 65% formamide for 30 s, which is followed by washing twice with DEPC-PBS-T. Detection probes that contained uracils an first treated with UNG treating buffer (1 phi29 polymerase buffer (Fermentas), 0.2 μg/μl BSA, 0.02 U/μl UNG (Fermentas)) for 10 min and washed twice with DEPC-PBS-T before the formamide incubation. A mix containing 500 nM of corresponding anchor primers in 2×SSC and 20% formamide is then added to the sample and incubated at RT for 30 min, and the incubation is followed by two brief washes with DEPC-PBS-T A ligation mix containing each interrogation probe, 1× T4 ligase buffer (Fermentas), 1 mM ATP (Fermentas) and 0.1 U/μl of T4 ligase (Fermentas) is applied to the samples and incubated for 30 min at RT. The unligated probes ar washed away by 3×1 min incubation with DEPC-PBS-T. The slides are mounted in Vectashield mounting medium containing 100 ng/ml of DAPI for counterstaining the nuclei. The concentration of each interrogation probe is 100 nM for cell cultures and 500 nM for tissue sections. After imaging, the slides are prepared for the next sequencing cycle by UNG treatment buffer as described above followed by repeating the hybndization, ligation and imaging processes. To sequence each base, the same procedures are applied. For evaluation of loss of sequencing substrates (RCA products), cell cytoplasm is stained after the last sequencing cycle with Alexa Fluor 488 phalloidin (Invitrogen) in PBS at a final concentration of 0.15 μM, and the cells are incubated for 10 min at RT.

### APPENDIX 6: EXAMPLE OF SAMPLE STAINING FOR HIGH-CONTENT IMAGE ANALYSIS

Protocols from Gustafsdottir S M et al., 2013 may be used for sample staining for high-content image analysis. For example, samples may be stained as follows.

Step 1: MitoTracker and Wheat Germ Agglutinin Staining.

MitoTracker Deep Red (0M22426, Invitrogen) is dissolved in DMSO to 1 mM. Wheat Germ Agglutinin (WGA) Alexa594 conjugate (#WI1262, Invitrogen) is dissolved in dH2O to 1 mg/mL. A 500 nM MitoTracker, 60 μg/mL VGA solution is prepared in prewarmed media (DMEM, 10% FBS, 1% penicillin/streptomycin). Media is removed from plates; residual volume is 10 μL in each well. 30 μL of staining solution is added to wells and incubated for 30 min at 37° C.

Step 2: Fixation.

10 μL of 16% methanol-free paraformaldehyde (#15710-S, Electron Micrscopy Services) is added to wells for a final concentration of 3.2%. The plates are then incubated at room temperature for 20 min. Wells are washed once with 70 μL 1×HBSS (i14065-056, Invitrogen).

Step 3: Permeabilization.

A 0.1% solution of Triton X-100 (T8787-100 mL, Sigma) is prepared in 1×HBSS 30 μL of the solution is added to the wells and incubated for 10-20 min Wells are washed twice with 70 μL 1× I-IBSS.

Step 4: Phalloidin, ConcanavalinA, Hoechst, and SYTO 14 staining.

Concanavalin A Alexa488 conjugate (#C11252. Invitrogen) is dissolved to 1 mg/mL in 0.1 M sodium bicarbonate (SH30033.01. HyClone), and Phalloidin Alexa594 conjugate (#A12381. Invitrogen) is dissolved in 1.5 mL methanol (67-56-1, BDH) per vial A 0.025 μL phalloidin/IL solution, 100 μg/mL ConcanavalinA, 5 μg/mL Hoechst33342 (#H3570. Invitrogen), and 3 μM SYTO14 green fluorescent nucleic acid stain (#S7576. Invitrogen) solution is prepared in 1×HBSS, 1% BSA. 30 μL of staining solution is added to wells and incubated for 30 min. Wells ware washed three times with 70 μL 1×HBSS, no final aspiration. Plates are sealed with blue Remp thermal seal, at 171° C. for 4 s.

### Embodiments

Aspects of the present disclosure are directed to a method of altering cells with a pooled library of barcoded nucleic acids including combining a group of cells with the pooled library of bar coded nucleic acids under conditions which promote uptake of one or more barcoded nucleic acids from the pooled library into the cells, analyzing an individual cell for phenotype, sequencing the individual cell to identify the one or more barcoded nucleic acids from the pooled library, wherein the barcodes are functional or nonfunctional, and correlating the phenotype for the individual cell with the one or more barcoded nucleic acids from the pooled library. According to one aspect, the steps of analyzing, sequencing and correlating are conducted for multiple cells within the group of cells. According to one aspect, one or more barcoded nucleic acids from the pooled library are delivered to the cells using viral or non-viral methods. According to one aspect, the individual cell is analyzed for phenotype using automated imaging and one or more barcoded nucleic acids within the individual cell is sequenced using in situ sequencing. According to one aspect, the individual cell is analyzed for phenotype using automated imaging and one or more barcoded nucleic acids within the individual cell is sequenced using FISSEQ. According to one aspect, the cells are analyzed for phenotype using high-content imaged based analysis and one or more barcoded nucleic acids within the individual cell is sequenced using FISSEQ. According to one aspect, the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype. According to one aspect, the one or more barcoded nucleic acids are amplified in situ before using in situ sequencing. According to one aspect, the one or more barcoded nucleic acids are amplified in situ using isothermal or non-isothermal amplification before using in situ sequencing. According to one aspect, the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing. According to one aspect, the cells are eukaryotic cells, prokaryotic cells, animal cells, plant cells, yeast cells, fungal cells, bacteria cells, archaeal cells, or eubacterial cells. According to one aspect, the cells are mammalian cells. According to one aspect, the cells are human cells. According to one aspect, the cells are stem cells, pluripotent stem cells, or human induced pluripotent stem cells. According to one aspect, the cells are of a human organoid, an engineered human organ, an engineered organoid, an embryoid body or a whole organism.

The disclosure provides a method of altering cells with a pooled library of drugs including combining a group of cells with the pooled library of drugs under conditions which promote uptake of one or more drugs from the pooled library into the cells, analyzing an individual cell for phenotype, analyzing the individual cell to identify the one or more drugs from the pooled library, and correlating the phenotype for the individual cell with the one or more drugs from the pooled library. According to one aspect, the cells are eukaryotic cells, prokaryotic cells, animal cells, plant cells, yeast cells, fungal cells, bacteria cells, archaeal cells, or eubacterial cells. According to one aspect, the cells are mammalian cells. According to one aspect, the cells are human cells. According to one aspect, the cells are stem cells, pluripotent stem cells, or human induced pluripotent stem cells. According to one aspect, the cells are of a human organoid, an engineered human organ, an engineered organoid, an embryoid body or a whole organism.

The disclosure provides a method of altering cells with a pooled library of nucleic acids having fluorescent barcodes including combining a group of cells with the pooled library of nucleic acids having fluorescent barcodes under conditions which promote uptake of one or more nucleic acids having fluorescent barcodes from the pooled library into the cells, analyzing an individual cell for phenotype, analyzing the individual cell to identify the fluorescent barcode, and correlating the phenotype for the individual cell with the fluorescent barcode.

The disclosure provides a method of altering stem cells with a pooled library of bar coded nucleic acids Including combining a group of stem cells with the pooled library of bar coded nucleic acids under conditions which promote uptake of one or more barcoded nucleic acids from the pooled library into the stem cells, culturing the stem cells into embryoid bodies or organoids, analyzing individual cells from the embryoid bodies or organoids for phenotype, sequencing the individual cells to identify the one or more barcoded nucleic acids from the pooled library, wherein the barcodes are functional or nonfunctional, and correlating the phenotype for the individual cells with the one or more barcoded nucleic acids from the pooled library. According to one aspect, one or more barcoded nucleic acids from the pooled library are delivered to the cells using viral or non-viral methods. According to one aspect, the individual cells are analyzed for phenotype using automated imaging and one or more barcoded nucleic acids within the individual cells is sequenced using in situ sequencing. According to one aspect, the individual cells are analyzed for phenotype using automated imaging and one or more barcoded nucleic acids within the individual cells is sequenced using FISSEQ. According to one aspect, the individual cells are analyzed for phenotype using high-content imaged based analysis and one or more barcoded nucleic acids within the individual cells is sequenced using in situ sequencing. According to one aspect, the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype. According to one aspect, the one or more barcoded nucleic acids are amplified in situ before using in situ sequencing. According to one aspect, the one or more barcoded nucleic acids are amplified in situ using isothermal or non-isothermal amplification before using in situ sequencing. According to one aspect, the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing.

The disclosure provides a method of altering cells of an organoid or embryoid body with a pooled library of bar coded nucleic acids including combining the organoid or embryoid body with the pooled library of bar coded nucleic acids under conditions which promote uptake of one or more barcoded nucleic acids from the pooled library into individual cells of the organoid or embryoid body, analyzing the individual cells of the organoid or embryoid body for phenotype, sequencing the individual cells of the organoid or embryoid body to identify the one or more barcoded nucleic acids from the pooled library, wherein the barcodes are functional or nonfunctional, and correlating the phenotype for the individual cells of the organoid or embryoid body with the one or more barcoded nucleic acids from the pooled library. According to one aspect, one or more barcoded nucleic acids from the pooled library are delivered to the individual cells of the organoid or embryoid body using viral or non-viral methods. According to one aspect, the individual cells of the organoid or embryoid body are analyzed for phenotype using automated imaging and one or more barcoded nucleic acids within the individual cells of the organoid or embryoid body is sequenced using in situ sequencing. According to one aspect, the individual cells of the organoid or embryoid body are analyzed for phenotype using high-content imaged based analysis and one or more barcoded nucleic acids within the individual cells of the organoid or embryoid body is sequenced using in situ sequencing. According to one aspect, the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype. According to one aspect, the one or more barcoded nucleic acids are amplified in situ before using in situ sequencing. According to one aspect, the one or more barcoded nucleic acids are amplified in situ using isothermal or non-isothermal amplification before using in situ sequencing. According to one aspect, the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing.

The disclosure provides a method of altering cells of a plurality of embryoid bodies with a pooled library of bar coded nucleic acids including combining the embryoid bodies which are placed within a grid with the pooled library of bar coded nucleic acids under conditions which promote uptake of one or more barcoded nucleic acids from the pooled library into individual cells of the embryoid bodies, analyzing the individual cells of the embryoid bodies for phenotype for developing organoids or systems of organs, sequencing the individual cells of the embryoid bodies to identify the one or more barcoded nucleic acids from the pooled library, wherein the barcodes are functional or nonfunctional, and correlating the phenotype for the individual cells of the embryoid bodies with the one or more barcoded nucleic acids from the pooled library.

The disclosure provides a method of altering cells of a plurality of embryoid bodies with a pooled library of bar coded nucleic acids including combining the embryoid bodies which are placed within a grid with the pooled library of bar coded nucleic acids under conditions which promote uptake of one or more barcoded nucleic acids from the pooled library into individual cells of the embryoid bodies, culturing the embryoid bodies to form organoids, analyzing individual cells of the organoids for phenotype, sequencing the individual cells of the organoids to identify the one or more barcoded nucleic acids from the pooled library, wherein the barcodes are functional or nonfunctional, and correlating the phenotype for the individual cells of the organoids with the one or more barcoded nucleic acids from the pooled library.

### REFERENCES

The following references are incorporated herein by reference in their entireties.
1. Buchser, W. et al. Assay Guidance Manual. (2004).
2. Bray, M. A. & Carpenter, A. Assay Guidance Manual. (2004).
3. Wang, T., Wei, J. J., Sabatini, D. M. & Lander, E. S. Genetic screens in human cells using the CRISPR-Cas9 system. Science 343, 80-84 (2014).
4. Gilbert, L. A. et al. Genome-Scale CRISPR-Mediated Control of Gene Repression and Activation. Cell 159, 647-661 (2014).
5. Shalem, O., Sanjana, N. E. & Zhang, F. High-throughput functional genomics using CRISPR-Cas9. Nat Rev Genet 16, 299-311 (2015).
6. Shalem, O. et al. Genome-scale CRISPR-Cas9 knockout screening in human cells. Science 343, 84-87(2014).
7. Agrotis, A. & Ketteler, R. A new age in functional genomics using CRISPR/Cas9 in arrayed library screening. Front Genet 6, 300 (2015).
8. Lee, J. H. et al. Fluorescent in situ sequencing (FISSEQ) of RNA for gene expression profiling in intact cells and tissues. Nat Protoc 10, 442-458 (2015).
9. Lee, J. H. et al. Highly multiplexed subcellular RNA sequencing in situ. Science 343, 1360-1363 (2014).
10. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
11. Ke, R. et al. In situ sequencing for RNA analysis in preserved tissue and cells. Nat Methods 10, 857-860 (2013).
12. Nayerossadat, N., Maedeh, T. & Ali, P. A. Viral and nonviral delivery systems for gene delivery. Adv Biomed Res 1, 27 (2012).
13. Carpenter, A. E. et al. CellProfiler: image analysis software for identifying and quantifying cell phenotypes. Genome Biol 7, R100 (2006).
14. Crosetto, N., Bienko, M. & van Oudenaarden, A. Spatially resolved transcriptomics and beyond. Nat Rev Genet 16, 57-66 (2015).
15. Larsson, C., Grundberg, I., Söderberg, O. & Nilsson, M. In situ detection and genotyping of individual mRNA molecules. Nat Methods 7, 395-397 (2010).
16. Weibrecht, I. et al. In situ detection of individual mRNA molecules and protein complexes or post-translational modifications using padlock probes combined with the in situ proximity ligation assay. Nat Protoc 8, 355-372 (2013).
17. Chen, R. et al. A Barcoding Strategy Enabling Higher-Throughput Library Screening by Microscopy. ACS Synth Biol 4, 1205-1216 (2015).
18. Bagasra, O. Protocols for the in situ PCR-amplification and detection of mRNA and DNA sequences. Nat Protoc 2, 2782-2795 (2007).
19. Mitra, R. D. & Church, G. M. In situ localized amplification and contact replication of many individual DNA molecules. Nucleic Acids Res 27, e34 (1999).
20. Konermann, S. et al. Genome-scale transcriptional activation by an engineered CRISPR-Cas9 complex. Nature 517, 583-588 (2015).
21. Harkness, T. et al. High-Content Imaging with Micropattemed Multiwell Plates Reveals Influence of Cell Geometry and Cytoskeleton on Chromatin Dynamics. Biotechnol J(2015). doi:10.1002/biot.201400756
22. Bao, G., Mitragotri, S. & Tong, S. Multifunctional nanoparticles for drug delivery and molecular imaging. Annu Rev Biomed Eng 15, 253-282 (2013).
23. Meinhardt, A. et al. 3D reconstitution of the patterned neural tube from embryonic stem cells. Stem Cell Reports 3, 987-999 (2014).
24. Mitra, R. D., Shendure, J., Olejnik, J., Edyta-Krzymanska-Olejnik & Church, G. M. Fluorescent in situ sequencing on polymerase colonies. Anal Biochem 320, 55-65 (2003).

1. A method of altering cells with a pooled library of barcoded nucleic acids comprising
combining a group of cells with the pooled library of bar coded nucleic acids under conditions which promote uptake of one or more barcoded nucleic acids from the pooled library into the cells,
analyzing an individual cell for phenotype,
sequencing the individual cell to identify the one or more barcoded nucleic acids from the pooled library, wherein the barcodes are functional or nonfunctional, and
correlating the phenotype for the individual cell with the one or more barcoded nucleic acids from the pooled library.

2. The method of claim 1 wherein the steps of analyzing, sequencing and correlating are conducted for multiple cells within the group of cells.

3. The method of claim 1 wherein one or more barcoded nucleic acids from the pooled library are delivered to the cells using viral or non-viral methods.

4. The method of claim 1 wherein the individual cell is analyzed for phenotype using automated imaging and one or more barcoded nucleic acids within the individual cell is sequenced using in situ sequencing.

5. The method of claim 1 wherein the individual cell is analyzed for phenotype using automated imaging and one or more barcoded nucleic acids within the individual cell is sequenced using FISSEQ.

6. The method of claim 1 wherein the cells are analyzed for phenotype using high-content imaged based analysis and one or more barcoded nucleic acids within the individual cell is sequenced using FISSEQ.

7. The method of claim 1 wherein the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype.

8. The method of claim 1 wherein the one or more barcoded nucleic acids are amplified in situ before using in situ sequencing.

9. The method of claim 1 wherein the one or more barcoded nucleic acids are amplified in situ using isothermal or non-isothermal amplification before using in situ sequencing.

10. The method of claim 1 wherein the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing.

11. The method of claim 1 wherein the cells are eukaryotic cells, prokaryotic cells, animal cells, plant cells, yeast cells, fungal cells, bacteria cells, archaeal cells, or eubacterial cells.

12. The method of claim 1 wherein the cells are mammalian cells.

13. The method of claim 1 wherein the cells are human cells.

14. The method of claim 1 wherein the cells are stem cells, pluripotent stem cells, or human induced pluripotent stem cells.

15. The method of claim 1 wherein the cells are of a human organoid, an engineered human organ, an engineered organoid, an embryoid body or a whole organism.

16. A method of altering cells with a pooled library of drugs comprising
combining a group of cells with the pooled library of drugs under conditions which promote uptake of one or more drugs from the pooled library into the cells,
analyzing an individual cell for phenotype,
analyzing the individual cell to identify the one or more drugs from the pooled library, and
correlating the phenotype for the individual cell with the one or more drugs from the pooled library.

17. The method of claim 16 wherein the cells are eukaryotic cells, prokaryotic cells, animal cells, plant cells, yeast cells, fungal cells, bacteria cells, archaeal cells, or eubacterial cells.

18. The method of claim 16 wherein the cells are mammalian cells.

19. The method of claim 16 wherein the cells are human cells.

20. The method of claim 16 wherein the cells are stem cells, pluripotent stem cells, or human induced pluripotent stem cells.

21. The method of claim 16 wherein the cells are of a human organoid, an engineered human organ, an engineered organoid, an embryoid body or a whole organism.

22. A method of altering cells with a pooled library of nucleic acids having fluorescent barcodes comprising
combining a group of cells with the pooled library of nucleic acids having fluorescent barcodes under conditions which promote uptake of one or more nucleic acids having fluorescent barcodes from the pooled library into the cells,
analyzing an individual cell for phenotype,
analyzing the individual cell to identify the fluorescent barcode, and
correlating the phenotype for the individual cell with the fluorescent barcode.

23. A method of altering stem cells with a pooled library of bar coded nucleic acids comprising
combining a group of stem cells with the pooled library of bar coded nucleic acids under conditions which promote uptake of one or more barcoded nucleic acids from the pooled library into the stem cells,
culturing the stem cells into embryoid bodies or organoids,
analyzing individual cells from the embryoid bodies or organoids for phenotype,
sequencing the individual cells to identify the one or more barcoded nucleic acids from the pooled library, wherein the barcodes are functional or nonfunctional, and
correlating the phenotype for the individual cells with the one or more barcoded nucleic acids from the pooled library.

24. The method of claim 23 wherein one or more barcoded nucleic acids from the pooled library are delivered to the cells using viral or non-viral methods.

25. The method of claim 23 wherein the individual cells are analyzed for phenotype using automated imaging and one or more barcoded nucleic acids within the individual cells is sequenced using in situ sequencing.

26. The method of claim 23 wherein the individual cells are analyzed for phenotype using automated imaging and one or more barcoded nucleic acids within the individual cells is sequenced using FISSEQ.

27. The method of claim 23 wherein the individual cells are analyzed for phenotype using high-content imaged based analysis and one or more barcoded nucleic acids within the individual cells is sequenced using in situ sequencing.

28. The method of claim 23 wherein the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype.

29. The method of claim 23 wherein the one or more barcoded nucleic acids are amplified in situ before using in situ sequencing.

30. The method of claim 23 wherein the one or more barcoded nucleic acids are amplified in situ using isothermal or non-isothermal amplification before using in situ sequencing.

31. The method of claim 23 wherein the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing.

32. A method of altering cells of an organoid or embryoid body with a pooled library of bar coded nucleic acids comprising
combining the organoid or embryoid body with the pooled library of bar coded nucleic acids under conditions which promote uptake of one or more barcoded nucleic acids from the pooled library into individual cells of the organoid or embryoid body,
analyzing the individual cells of the organoid or embryoid body for phenotype,
sequencing the individual cells of the organoid or embryoid body to identify the one or more barcoded nucleic acids from the pooled library, wherein the barcodes are functional or nonfunctional, and
correlating the phenotype for the individual cells of the organoid or embryoid body with the one or more barcoded nucleic acids from the pooled library.

33. The method of claim 32 wherein one or more barcoded nucleic acids from the pooled library are delivered to the individual cells of the organoid or embryoid body using viral or non-viral methods.

34. The method of claim 32 wherein the individual cells of the organoid or embryoid body are analyzed for phenotype using automated imaging and one or more barcoded nucleic acids within the individual cells of the organoid or embryoid body is sequenced using in situ sequencing.

35. The method of claim 32 wherein the individual cells of the organoid or embryoid body are analyzed for phenotype using high-content imaged based analysis and one or more barcoded nucleic acids within the individual cells of the organoid or embryoid body is sequenced using in situ sequencing.

36. The method of claim 32 wherein the phenotype is correlated with the one or more barcoded nucleic acids from the pooled library using a bioinformatic pipeline to merge cytometric data with sequence information at the single cell level to allow statistical deconvolution of the effect of a nucleic acid from the library with resulting phenotype.

37. The method of claim 32 wherein the one or more barcoded nucleic acids are amplified in situ before using in situ sequencing.

38. The method of claim 32 wherein the one or more barcoded nucleic acids are amplified in situ using isothermal or non-isothermal amplification before using in situ sequencing.

39. The method of claim 32 wherein the one or more barcoded nucleic acids are delivered using an expression vector where the nucleic acid is flanked by a first common sequence and a second common sequence and promoter which facilitates expression of RNA copies of the nucleic acid, wherein the RNA is reversed transcribed, circularized and amplified by rolling circle amplification before using in situ sequencing.

40. A method of altering cells of a plurality of embryoid bodies with a pooled library of bar coded nucleic acids comprising
combining the embryoid bodies which are placed within a grid with the pooled library of bar coded nucleic acids under conditions which promote uptake of one or more barcoded nucleic acids from the pooled library into individual cells of the embryoid bodies,
analyzing the individual cells of the embryoid bodies for phenotype for developing organoids or systems of organs,
sequencing the individual cells of the embryoid bodies to identify the one or more barcoded nucleic acids from the pooled library, wherein the barcodes are functional or nonfunctional, and
correlating the phenotype for the individual cells of the embryoid bodies with the one or more barcoded nucleic acids from the pooled library.

41. A method of altering cells of a plurality of embryoid bodies with a pooled library of bar coded nucleic acids comprising
combining the embryoid bodies which are placed within a grid with the pooled library of bar coded nucleic acids under conditions which promote uptake of one or more barcoded nucleic acids from the pooled library into individual cells of the embryoid bodies,
culturing the embryoid bodies to form organoids,
analyzing individual cells of the organoids for phenotype,
sequencing the individual cells of the organoids to identify the one or more barcoded nucleic acids from the pooled library, wherein the barcodes are functional or nonfunctional, and
correlating the phenotype for the individual cells of the organoids with the one or more barcoded nucleic acids from the pooled library.


SEQUENCE LISTING (6 sequences)

SEQ ID NO: 1 - DNA, 38 bp, Artificial Sequence
  Description of Artificial Sequence Synthetic primer
acttcagctgccccgggtgaagatgataacggactagc

SEQ ID NO: 2 - DNA, 38 bp, Artificial Sequence
  Description of Artificial Sequence Synthetic primer
acttcagctgccccgggtgaagactagccttattttaa

SEQ ID NO: 3 - DNA, 25 bp, Artificial Sequence
  Description of Artificial Sequence Synthetic probe
aacttgctatttctagctctaaaac

SEQ ID NO: 4 - DNA, 23 bp, Artificial Sequence
  Description of Artificial Sequence Synthetic oligonucleotide
gttttagagctagaaatagcaag

SEQ ID NO: 5 - DNA, 18 bp, Artificial Sequence
  Description of Artificial Sequence Synthetic oligonucleotide
tcttcagcgttcccgaga

SEQ ID NO: 6 - DNA, 200 bp, Artificial Sequence
  Description of Artificial Sequence Synthetic polynucleotide
tcatatgcttaccgtaacttgaaagtatttcgatttcttggctttatatatcttgtggaaaggacgaaacaccggagacgggataccgtctctgttttagagctaggccaacatgaggatcacccatgtctgcagggcctagcaagttaaaataaggctagtccgttatcaacttggccaacatgaggatcacccatgtc